(19) United States
(12) Patent Application Publication
(10) Pub. No.: US 2024/0041750 A1
(43) Pub. Date: Feb. 8, 2024

(54) HALOSCENT IN DRY SHAMPOO

(71) Applicant: Firmenich SA, Satigny (CH)

(72) Inventors: Glenn VERHOVNIK, Satigny (CH); Pauline LEDERREY, Satigny (CH)

(21) Appl. No.: 18/255,978

(22) PCT Filed: Dec. 1, 2021
(86) PCT No.: PCT/EP2021/083701

§ 371 Date: Jun. 5, 2023

(30) Foreign Application Priority Data
Dec. 3, 2020 (EP) 20211529.1

(51) Int. Cl. A61K 8/899; A61Q 5/02; A61Q 13/00; A61K 8/46

(52) U.S. Cl. CPC A61K 8/899; A61Q 5/02; A61Q 13/00; A61K 8/46

(57) ABSTRACT

The present invention relates to a dry shampoo composition comprising a perfume composition comprising a pro-perfume compound and a dry shampoo active material, a dry shampoo article comprising the same and methods and uses of the same for conferring, improving, enhancing, modifying or prolonging the fragrance intensity of the perfume composition.

### TECHNICAL FIELD OF THE INVENTION

The present invention relates to a dry shampoo composition comprising a perfume composition comprising a pro-perfume compound and a dry shampoo active material, a dry shampoo article comprising the same and methods and uses of the same for conferring, improving, enhancing, modifying or prolonging the fragrance intensity of the perfume composition.

### BACKGROUND OF THE INVENTION

The perfume industry has a particular interest for compositions or additives, which are capable of prolonging or enhancing the perfuming effect of at least one perfuming ingredient for a certain period of time. It is particularly desirable to obtain long-lasting properties for standard perfumery raw materials which are too volatile or have a poor substantivity by themselves, or which are only deposited in a small amount onto the surface of the final application. Furthermore, some of the perfumery ingredients are unstable and need to be protected against slow degradation prior to their use. Long-lasting perfumes are desirable for various applications, as for example for cleaning hair in which there is a constant need to enable the effect of active substances, in particular perfumes, or perfuming compositions, to be effective for a certain period of time after application. Indeed, many active substances which are particularly suitable for this type of application are known to lack tenacity on hair, or do not remain on the thereon, with the result that their perfuming effect is experienced only briefly and not very intensely. Given the importance of this type of application in the perfume industry, research in this field has been sustained, in particular with the aim of finding new, and more effective solutions to the aforementioned problems.

It has now been surprisingly found that a dry shampoo composition according to the present invention solves the above-mentioned problems, and is capable of efficiently releasing fragrance compounds (perfumery raw materials) from pro-perfume compounds comprised in the dry shampoo composition. In particular, it has been surprisingly found that the dry shampoo composition results in a more efficient and prolonged release of fragrance compounds when applied to hair.

### DESCRIPTION OF THE FIGURES

FIG. 1: Comparison of the fragrance intensity of a dry shampoo composition according to the invention with a dry shampoo composition not according to the invention after 24 h and 48 h after application on hair.

### DETAILED DESCRIPTION OF THE INVENTION

The present invention relates to a dry shampoo composition comprising
a perfume composition comprising a pro-perfume compound,
a dry shampoo active material and
optionally, a carrier material.

A dry shampoo composition is understood as a composition that is effective in cleaning hair without the necessity for rinsing with water following its application. A dry shampoo composition can be applied to hair and then optionally be brushed through the hair with the fingers or a hair utensil, such as a comp or brush, to affect the cleansing effect. In contrast to dry shampoo compositions, wet shampoo compositions are understood to be applied to wet hair, lathered and then rinsed with added water. When using a dry shampoo composition, no rinsing with water is required.

The dry shampoo composition according to the present invention comprises a perfume composition.

A perfume composition as herein described is a composition that is able to impart a hedonic effect to e.g. a consumer product. In other words, a composition to be considered as being a perfume composition must be recognized by a skilled person in the art of perfumery as being able to impart or modify the olfactory perception in a positive or pleasant way, and not just as imparting an odor.

The perfume composition comprises at least one perfuming ingredient or a mixture of perfuming ingredients. The perfuming ingredient is not a pro-perfume compound according to the invention. A perfuming ingredient is a compound, which is used in a perfuming preparation or composition to impart a hedonic effect. In other words such a perfuming ingredient, to be considered as being a perfuming one, must be recognized by a person skilled in the art as being able to impart or modify in a positive or pleasant way the odor of a composition, and not just as having an odor.

The nature and type of the perfuming ingredients do not warrant a more detailed description here, which in any case would not be exhaustive, the skilled person being able to select them on the basis of general knowledge and according to intended use or application and the desired organoleptic effect. In general terms, these perfuming ingredients belong to chemical classes as varied as alcohols, lactones, aldehydes, ketones, esters, ethers, acetates, nitriles, terpene hydrocarbons, nitrogenous or sulphurous heterocyclic compounds and essential oils, and the perfuming ingredients can be of natural or synthetic origin. In particular, one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients:methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2- ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)- 2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxo1-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

A perfume composition may not be limited to the above mentioned perfuming ingredients, and many other of these ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

The perfume composition may further comprise a solvent. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyltriacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate, ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF), or mixtures thereof, which are the most commonly used.

The perfume composition may further comprise an adjuvant. By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

The perfume composition may further comprise at least on solid carrier. Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture threof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

In a particular embodiment, the dry shampoo composition comprises the perfume composition in an amount of from 0.001 wt. % to 10 wt. %, preferably from 0.01 wt. % to 5 wt. %, preferably from 0.05 wt. % to 2.5 wt. %, preferably from 0.07 wt. % to 1.5 wt. %, preferably from 0.1 wt. % to 0.5 wt. %, based on the total weight of the dry shampoo composition.

The perfume composition of the dry shampoo composition according to the present invention comprises a pro-perfume compound.

A pro-perfume compound or pro-fragrance compound, respectively, as used herein refers to a structure, which links a perfume compound, also termed PRM (perfumery raw material), via a covalent bond to a suitable substrate to form a non-volatile perfume conjugate. Non-volatile perfume conjugate as used herein is understood in that the pro-perfume compound is a perfume conjugate, which is less volatile as the perfume compound or even not volatile under ambient conditions. In other words, the pro-fragrance compound is a compound that is able to release one, two, or three perfumery raw material(s) upon cleavage of a chemical bond/chemical bonds.

This conjugate provides a release of a perfume compound, in particular an olfactive PRM compound, upon trigger with an external stimulus. An external stimulus may be the contact with moisture and/or exposure to light and/or increased temperature and/or oxidative environment.

The perfume composition may comprise pro-perfume compounds that release PRMs upon exposure to light.

The perfuming composition may comprise pro-perfume compounds that release PRMs upon exposure to air/oxygen. Thereby, it is understood that the PRMs may be released from such pro-perfumes by oxidation in the presence of air (ambient air) or oxygen, preferably ambient air. For the sake of clarity, by the expression “ambient air”, or the similar, it is meant the normal meaning understood by a person skilled in the art, i.e. the oxidation occurs at room temperature, under air and atmospheric pressure. In other words, the environment wherein the compound is oxidized is air. Herewith it is understood, that the pro-perfume is oxidized in ambient air. In particular, it is understood that pro-perfume does not necessarily require a pure oxygen environment, heat or catalyst to be oxidized.

The perfuming composition according to the present invention may comprise pro-perfume compounds that release PRMs upon exposure to moisture. Such pro-perfumes may show chemical bonds that are susceptible to water-induced cleavage, and may thus be cleaved in the presence of water.

In a particular embodiment, the pro-perfume compound that release PRMs upon exposure by air/oxygen and/or moisture, preferably the pro-perfume compound releases PRMs upon exposure by air/oxygen.

Pro-perfume compounds may be based on the Michael-type 1,4-addition of amines, alcohols, carboxylic acids and thiols reacting with the double bond of α,β-unsaturated ketones of a perfume compound. The perfume compounds provide a certain control of the kinetics of fragrance release to induce sensory effects through sequential release. In particular, oxygen is used as external stimulus for the release of the perfume compound.

In a particular embodiment, the pro-perfume compound is a compound of formula

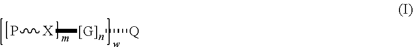

wherein:
a) w represents an integer from 1 to 10000;
b) n represents 1 or 0;
c) m represents an integer from 1 to 6;
d) P represents a hydrogen atom or a radical susceptible of generating an odoriferous α,β-unsaturated ketone, aldehyde or carboxylic ester and is represented by the formula

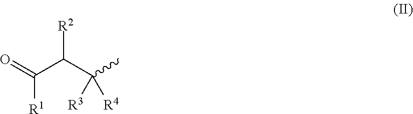

in which the wavy line indicates the location of the bond between said P and X; R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups; and with the proviso that at least one of the P groups is of the formula (II) as defined hereinabove;
e) X represents, independently from each other, a functional group selected from the group consisting of the formulae i) to xiv):

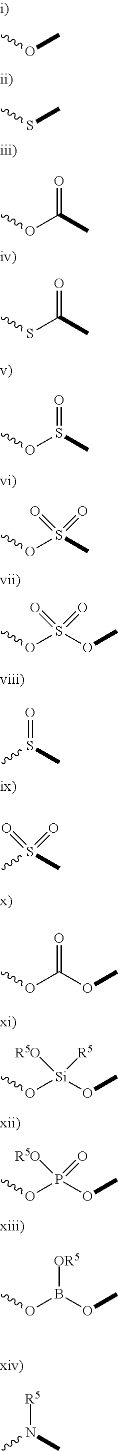

in which formulae the wavy lines are as defined previously and the bold lines indicate the location of the bond between said X and G, and R⁵ represents a hydrogen atom, a C₁ to C₂₂, saturated or unsaturated, alkyl group or an aryl group, possibly substituted by C₁ to C₆ alkyl or alkoxyl groups or halogen atoms; and with the proviso that X may not exist when P represents a hydrogen atom;
f) G represents a multivalent radical (with a m+1 valence) derived from cyclic, linear, alicyclic or branched alkyl, cyclic, linear, alicyclic or branched alkenyl, phenyl, alkylphenyl or alkenylphenyl hydrocarbon radical having from 1 to 22 carbon atoms, said hydrocarbon radical being possibly substituted and containing from 1 to 10 functional groups selected from the group consisting of halogens, alcohols, ethers, esters, ketones, aldehydes, carboxylic acids, thiols, thioethers, amines, quaternary amines and amides; and
g) Q represents a hydrogen atom (in which case w=1 and n=1), or represents a polymer or co-polymer selected from the group consisting of poly(alkylimine)s, polypeptides (e.g. polylysine) or polysaccharides selected from the group consisting of cellulose, cyclodextrins and starches, or cationic quaternized silicon polymers, or still a polymer or random co-polymer derived from monomeric units selected from the group consisting of the formulae A-1), A-2),

A-3), B-1), B-2), C-1), C-2), and C-3):

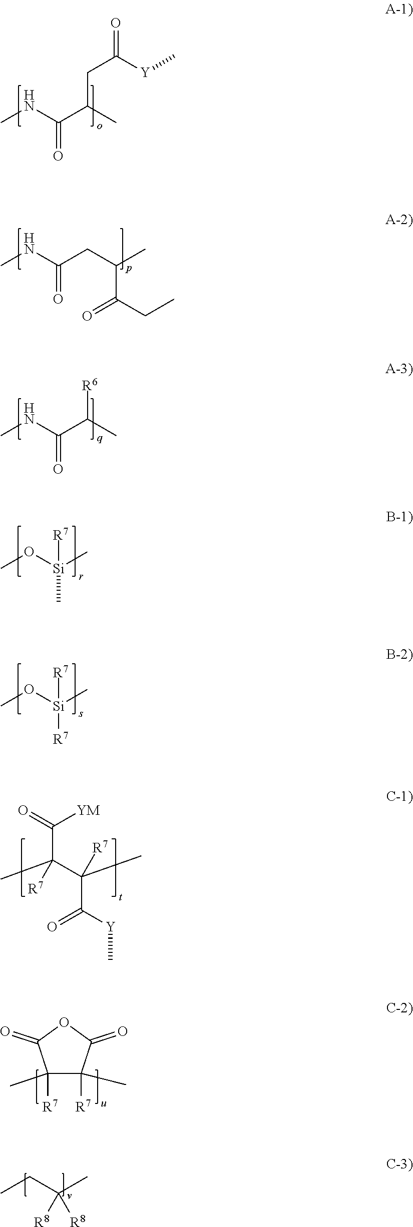

wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid, such as glycine, alanine, phenylalanine, arginine, histidine, lysine, aspartic acid, glutamic acid, cysteine, methionine, glutamine, asparagine, threonine, serine, leucine, isoleucine, valine, tyrosine or tryptophan;
R⁷ represents, simultaneously or independently, a hydrogen atom or a C₁-C₁₆ hydrocarbon group;
R⁸ represents, simultaneously or independently of each other
   a hydrogen or halide atom;
   a C₁-C₆ hydrocarbon group optionally comprising from 1 to 4 heteroatoms selected from the group consisting of oxygen and sulfur atoms;
   a carboxylic group of formula COOR*, wherein R* represents a hydrogen atom, a C₆₀ alkyl or alkenyl group optionally comprising from 1 to 30 oxygen atoms;
   a OR⁷ group or a COR⁷ group; or
   a pyrrolidone unit, connected by the nitrogen atom; and
M represents a hydrogen atom, an alkali or earth alkali metal ion.

In a particular embodiment, X represents a functional group selected from the group consisting of the formulae ii), iii), viii), ix) and xiv). In a particular embodiment, X represents the functional group of formula ii).

As “odoriferous α,β-unsaturated ketone, aldehyde or carboxylic ester”, the expression used in the definition of P, it is understood an α,β-unsaturated ketone, aldehyde or carboxylic ester, which is recognized by a skilled person as being used in perfumery as perfuming ingredient.

In general, said odoriferous α,β-unsaturated ketone, aldehyde or carboxylic ester is a compound having from 8 to 20 carbon atoms, or even more preferably between 10 and 15 carbon atoms.

Similarly, it is not possible to provide an exhaustive list of the currently known odoriferous compounds, which can be used in the synthesis of the invention compounds defined hereinabove and subsequently be released. However, the following can be named as preferred examples: alpha-damascone, beta-damascone, gamma-damascone, delta-damascone, alpha-ionone, beta-ionone, gamma-ionone, delta-ionone, beta-damascenone, 1-[6-ethyl-2,6-dimethyl-3-cyclohexen-1-yl]-2-buten-1-one, 2-methyl-1-(2,6,6-trimethylcyclohex-3-en-1-yl)but-2-en-1-one, 3-methyl-5-propyl-2-cyclohexen-1-one, 2-methyl-5-(1-propen-2-yl)-2-cyclohexen-1-one, 2,5-dimethyl-5-phenyl-1-hexen-3-one, 1-(5,5-dimethyl-1-cyclohexen-1-yl)-4-penten-1-one, 3,7-dimethylocta-2,6-dienal, 8-methyl-alpha-ionone or 10-methyl-alpha-ionone, 2-octenal, 1-(2,2,3,6-tetramethylcyclohexyl)but-2-en-1-one, 4-(2,2, 3,6-tetramethylcyclohexyl)but-3-en-2-one, 2-cyclopentadecen-1-one, 4,4a-dimethyl-6-(1-propen-2-yl)-4,4a,5,6,7,8-hexahydro-2(3H)-naphthalenone, (E)-3-phenylprop-2-enal (cinnamic aldehyde), 2,6,6-trimethylspiro[bicyclo[3.1.1]heptane-3,1′-cyclohexan]-2′-en-4′-one, ethyl 2,4-deca-dienoate, ethyl 2-octenoate, methyl 2-nonenoate, ethyl 2,4-undecadienoate, 4-methylpent-3-en-2-one, oct-2-en-4-one, and methyl 5,9-dimethyl-2,4,8- decatrienoate.

In a particular embodiment, P represents a radical selected from the group consisting of formulae (P-1) to (P-14), in the form of any one of its isomers:

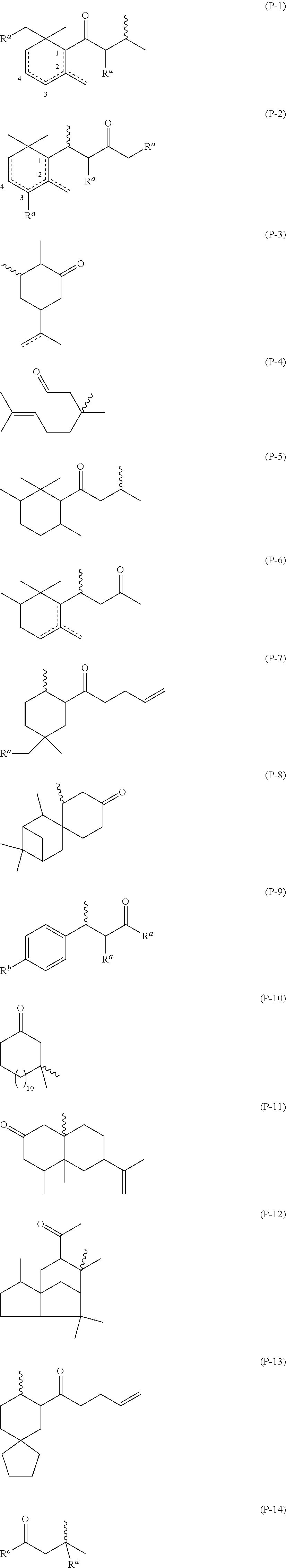

in which formulae the wavy lines have the meaning indicated above and the dotted lines represent a single or double bond, R a being a hydrogen atom or a methyl group and R b representing a hydrogen atom, a hydroxyl or methoxy group or a C₁-C₄ linear or branched alkyl group and R^{c} representing a hydrogen atom or a C₁-C₄ linear or branched alkyl group.

In a particular embodiment, P represents a radical selected from the group consisting of formulae

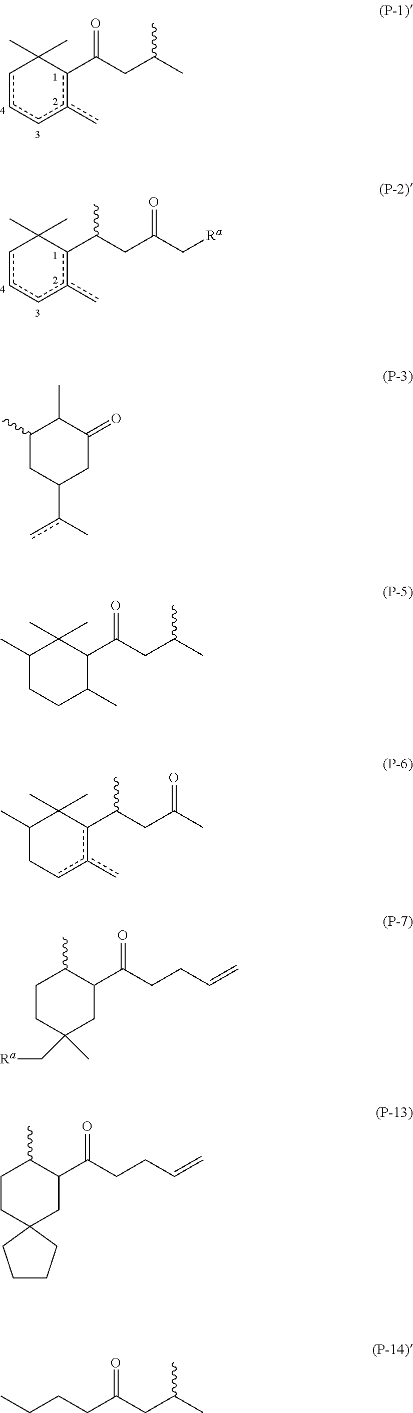

wherein the wavy lines have the meaning indicated above and the dotted lines represent a single or double bond, and R a being a hydrogen atom or a methyl group.

In a particular embodiment, P represents a radical selected from the group consisting of formulae (P-1), (P-2), (P-1)′, (P-2)′, (P-3), (P-7), (P-13), (P-14) or (P-14)′ as defined above. Preferably, P represents a radical selected from the group consisting of formulae (P-1), (P-1)′, (P-2), (P-2)′, (P-3) or (P-14)′ as defined above.

In a particular embodiment, G may represent a divalent cyclic, linear, alicyclic or branched alkyl, alkenyl, alkandienyl or alkylbenzene hydrocarbon radical having from 1 to 22 carbon atoms, said hydrocarbon radical being possibly substituted and containing from 1 to 10 functional groups selected from the group consisting of ethers, esters, ketones, aldehydes, carboxylic acids, thiols, thioethers, amines, quaternary amines and amides.

In a particular embodiment, G may represent a divalent linear or branched alkyl or alkenyl hydrocarbon radical having from 1 to 22 carbon atoms, said hydrocarbon radical being possibly substituted and containing from 1 to 5 functional groups selected from the group consisting of ethers, esters, ketones, aldehydes, carboxylic acids, thiols, thioethers, amines, quaternary amines and amides.

In a particular embodiment, G may represent a divalent linear or branched alkyl or alkenyl hydrocarbon radical having from 2 to 15 carbon atoms, said hydrocarbon radical being possibly substituted and containing from 1 to 2 functional groups selected from the group consisting of ethers and esters.

In a particular embodiment, G may represent a divalent linear alkyl or alkenyl hydrocarbon radical having from 3 to 15 carbon atoms, said hydrocarbon radical being possibly substituted and containing one ester functional group.

In a particular embodiment, G may represent a divalent linear alkyl or alkenyl hydrocarbon radical having from 3 to 14 carbon atoms.

In a particular embodiment, Q may represent a hydrogen atom or a co-polymer comprising at least one repeating unit of formula B-1 as defined above.

In a particular embodiment, Q may represent a hydrogen atom or a co-polymer comprising at least one repeating unit of formula B-1 and at least one repeating unit of formula B-2.

In a particular embodiment, R⁷ may represent, simultaneously or independently, a hydrogen atom or a C₁₋₃ alkyl group. Preferably, R⁷ may represent, simultaneously or independently, a hydrogen atom or a methyl or an ethyl group. More preferably, R⁷ may represent, simultaneously or independently, a hydrogen atom or a methyl group.

In a particular embodiment, the pro-perfume compound is defined by formula (I) as mentioned above, wherein
w=1; n=1; m=1;
P represents a radical susceptible of generating an odoriferous α,β-unsaturated ketone, aldehyde and is represented by the formula

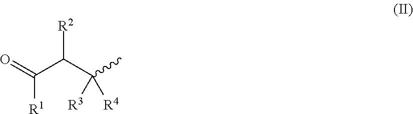

wherein R², R³ and R⁴ represent, independently of each other, a hydrogen atom, a C₆ to C₁₀ aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
X represents formula ii)
G represents a divalent radical derived from cyclic, linear or branched alkyl, alkenyl, phenyl, alkylphenyl or alkenylphenyl hydrocarbon radical having from 2 to 8 carbon atoms optionally comprising 1 or 2 oxygen, sulfur and/or nitrogen atoms
Q represents a polymer or random co-polymer derived from formula B-1), wherein R⁷ represents a C₁ -C₁₆ hydrocarbon group.

In a particular embodiment, the sulfur-containing pro-perfume compound is of formula (IV)

P—S-G-Q (IV)

wherein
P has the same meaning as defined above:
G represents a divalent radical derived from a linear or branched alkyl or alkenyl radical having from 2 to 15 carbon atoms, possibly substituted with one or more groups selected from the group consisting of —OR⁹, —N(R⁹)₂, —COOR⁹ and R⁹ groups, in which R⁹ represents a hydrogen atom or a C₁ to C₆ alkyl or alkenyl group; and
Q represents a hydrogen atom.

In a particular embodiment, the pro-perfume compound is a compound selected from the group consisting of formulae a) to d)

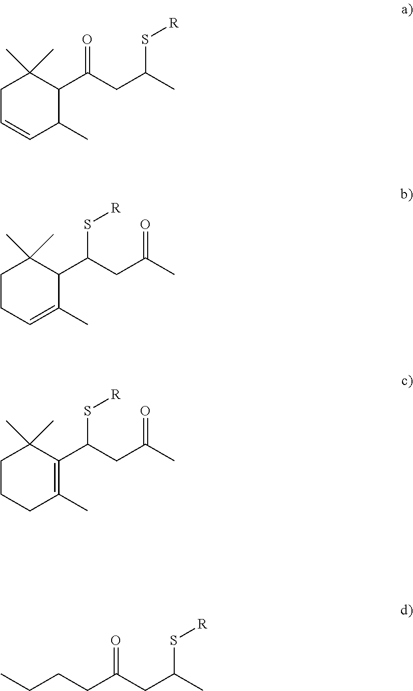

wherein R represents a C₁-C₂₀ alkyl or alkenyl group, preferably a C₆ -C₁₆ alkyl or alkenyl group, more preferably a C₁₂ alkyl group.

The pro-perfume of formula a) releases delta-damascone as fragrance compound. Said pro-perfume may preferably be (±)-trans-3-(dodecylthio)-1-(2,6,6-trimethyl-3-cyclohexen-1-yl)-1-butanone. Delta-damascone is also known as 1-[(1RS,2SR)-2,6,6-trimethyl-3-cyclohexen-1-yl]-2-buten-1-one.

The pro-perfume of formula b) or c) releases ionone as fragrance compound. Said pro-perfume may be present as an isomeric mixture of formula b) and formula c). The isomeric mixture may have a weight ratio of formula b) and formula c) from 40:60 to 60:40. In particular, the isomeric mixture may have a weight ratio of formula b) and formula c) of about 55:45. In particular, said pro-perfume releases two isomers of ionone as fragrance compound.

In particular, the pro-perfume of formula b) releases alpha-ionone as fragrance compound. Said pro-perfume of formula b) may preferably be (±)-4-(dodecylthio)-4-(2,6,6-trimethyl-2-cyclohexen-1-yl)-2-butanone. Alpha-ionone is also known as (±)-(3E)-4-(2,6,6-trimethyl-2-cyclohexen-1-yl)-3-buten-2-one.

In particular, the pro-perfume of formula c) releases beta-ionone as fragrance compound. Said pro-perfume of formula c) may preferably be (±)-4-(dodecylthio)-4-(2,6,6-trimethyl-1-cyclohexen-1-yl)-2-butanone. Beta-ionone is also known as (3E)-4-(2,6,6-trimethyl-1-cyclohexen-1-yl)-3-buten-2-one.

The pro-perfume of formula d) releases oct-2-en-4-one as fragrance compound. Said pro-perfume may preferably be (±)-2-(dodecylthio)octan-4-one. Oct-2-en-4-one may be released as its (E)- or (Z)- isomers, or as mixtures thereof, with the (E)-isomer being preferred.

In a particular embodiment, the pro-perfume compound is a linear polysiloxane co-polymer comprising at least one repeating unit of formula

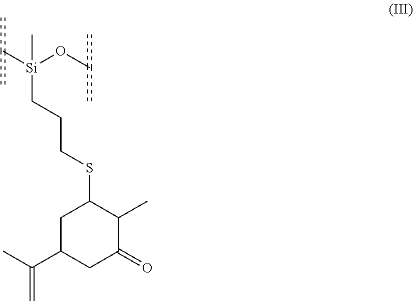

wherein the double hatched lines indicate the bonding to another repeating unit.

The pro-perfume of formula (III) releases 2-methyl-5-(prop-1-en-2-yl)cyclohex-2-en-1-one as fragrance compound, which is also known as carvone. Carvone exists in the form of two enantiomers, namely (R)-(−)-2-methyl-5-(1-propen-2-yl)-2-cyclohexen-1-one (L-carvone or carvone laevo) and (S)-(+)-2-methyl-5-(1-propen-2-yl)-2-cyclohexen-1-one (D-carvone or carvone dextro). The two enantiomers have been reported to have slightly different mint odor tonalities. According to any embodiment of the invention, both enantiomers are expected to have a similar effect in view of the preparation of the co-polymer and the release efficiency. According to the invention, carvone can either be used as a racemate or as a mixture enriched in either one of the two enantiomers. Preferably, a mixture enriched in carvone laevo is used.

In a particular embodiment, the pro-perfume compound of formula (I) may be selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1- yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4- ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 3-(dodecylsulfonyI)-1-(2,6,6-trimethylcyclohex-3-en-1-yl)butan-1-one, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, 2-(dodecylthio)-4-octanone, 2-(dodecylsulfonyl)octan-4-one, 4-(dodecylthio)-4-methylpentan-2-one, methyl or ethyl N,S-bis(4-oxo-4-(2,6,6-trimethylcyclohex-3-en-1-yl)butan-2-yl)-L-cysteinate, methyl or ethyl S-(4-oxo-4-(2,6,6-trimethylcyclohex-3-en-1-yl)butan-2-yl)-L-cysteinate, 4-oxooctan-2-yl dodecanoate, and any mixtures thereof. Preferably, the pro-perfume compound of formula (I) may be selected from the group consisting of 3-(dodecylthio)-1-(2,6,6-trimethylcyclohex-3-en-1-yl)butan-1-one (Haloscent® D), 3-(dodecylthio)-1-(2,6,6-trimethylcyclohex-2-en-1-yl)butan-1-one, 3-(dodecylsulfonyl)-1-(2,6,6-trimethylcyclohex-3-en-1-yl)butan-1-one, 4-(dodecylthio)-4-(2,6,6-trimethylcyclohex-2-en-1-yl)butan-2-one (Haloscent® I) and 4-(dodecylthio)-4-(2,6,6-trimethylcyclohex-1-en-1-yl)butan-2-one (Haloscent® I), 2-(dodecylthio)-4-octanone, 2-(dodecylsulfonyl)octan-4-one, 4-(dodecylthio)-4-methylpentan-2-one, methyl or ethyl N,S-bis(4-oxo-4-(2,6,6-trimethylcyclohex-3-en-1-yl)butan-2-yl)-L-cysteinate, methyl or ethyl S-(4-oxo-4-(2,6,6-trimethylcyclohex-3-en-1-yl)butan-2-yl)-L-cysteinate and 4-oxooctan-2-yl dodecanoate, or any mixtures thereof. Preferably, the pro-perfume compound of formula (I) may be 3-(dodecylthio)-1-(2,6,6-trimethylcyclohex-3-en-1-yl)butan-1-one (HaloScent® D), 4-(dodecylthio)-4-(2,6,6-trimethylcyclohex-2-en-1-yl)butan-2-one (HaloScent® I), 4-(dodecylthio)-4-(2,6,6-trimethylcyclohex-1-en-1-yl)butan-2-one (HaloScent® I), or a mixture thereof. Preferably, the pro-perfume compound of formula (I) may be a mixture of 3-(dodecylthio)-1-(2,6,6-trimethylcyclohex-3-en-1-yl)butan-1-one (HaloScent® D), 4-(dodecylthio)-4-(2,6,6-trimethylcyclohex-1-en-1-yl)butan-2-one (HaloScent® I) and 4-(dodecylthio)-4-(2,6,6-trimethylcyclohex-2-en-1-yl)butan-2-one (HaloScent® I).

In a particular embodiment, the pro-perfume compound is selected from the group consisting of 3-(dodecylthio)-1-(2,6,6-trimethylcyclohex-3-en-1-yl)butan-1-one (Haloscent® D), 2-(dodecylthio)-4-octanone, 2-(dodecylsulfonyl)octan-4-one, 3-(dodecylthio)-1-(2,6,6-trimethylcyclohex-2-en-1-yl)butan-1-one, 4-(dodecylthio)-4-(2,6,6-trimethylcyclohex-2-en-1-yl)butan-2-one (Haloscent® I), and 4-(dodecylthio)-4-(2,6,6-trimethylcyclohex-1-en-1-yl)butan-2-one (Haloscent® I), 4-(dodecylthio)-4-methylpentan-2-one, methyl or ethyl N,S-bis(4-oxo-4-(2,6,6-trimethylcyclohex-3-en-1-yl)butan-2-yl)-L-cysteinate and 4-oxooctan-2-yl dodecanoate, or any mixtures thereof.

According to any embodiments of the invention, the perfume composition may, optionally, comprises one or more additional profragrances selected from the group consisting of 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-(3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde, and any mixture thereof.

In a particular embodiment, the perfume composition comprises the pro-perfume compound in an amount of from 0.1 wt. % to 30 wt. %, preferably from 0.5 wt. % to 25 wt. %, preferably from 1 wt. % to 20 wt. %, based on the total weight of the perfume composition.

In a particular embodiment, the dry shampoo composition comprises the pro-perfume compound in an amount of from 0.0001 wt. % to 10 wt. %, preferably from 0.001 wt. % to 3 wt. %, preferably from 0.01 wt. % to 2 wt. %., preferably from 0.015 wt. % to 1 wt. %, based on the total weight of the dry shampoo composition.

The dry shampoo composition according to the present invention comprises a dry shampoo active material.

A dry shampoo active material is herein understood as a dry shampoo cleaning agent for inter alia removing and/or absorbing oil, grease, sebum and/or other undesired contaminants from hair.

In a particular embodiment, the dry shampoo active material comprises a sebum, grease and/or oil absorbing material.

In a particular embodiment, the dry shampoo active material comprises a starch material, preferably a natural starch material, modified starch material, preferably a chemically modified starch material, such as by esterification, etherification, oxidation, acid hydrolysis or cross-linking, or enzymatically modified starch material, or any combination thereof.

In a particular embodiment, the dry shampoo active material comprises a natural or unmodified starch material, respectively, selected from the group consisting of corn starch, potato starch, tapioca starch, rice starch, wheat starch, cassaya starch or any combination thereof, preferably corn starch, rice starch and tapioca starch or any combination thereof.

In a particular embodiment, the dry shampoo active material comprises a modified starch material selected from the group consisting of aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch, and sodium starch glycolate or any combination thereof, preferably aluminum starch octenylsuccinate.

In a particular embodiment, the dry shampoo active material comprises a natural or unmodified starch material and a modified starch material.

In a particular embodiment, the dry shampoo composition comprises the dry shampoo active material in an amount of from 15 wt. % to 55 wt. %, from 20 wt. % to 50 wt. %, from 25 wt. % to 45 wt. %, from 30 wt. % to 40 wt. %, based on the total weight of the dry shampoo composition.

In a particular embodiment, the dry shampoo composition may comprise a carrier material.

In a particular embodiment, the carrier may be a solvent, preferably a cosmetically acceptable solvent.

In a particular embodiment, the carrier may be ethanol, isopropanol or any combination thereof.

In a particular embodiment, the dry shampoo composition comprises the carrier in an amount of from 40 wt. % to 85 wt. %, preferably from 50 wt. % to 75 wt. %, more preferably from 60 wt. % to 65 wt. %, based on the total weight of the dry shampoo composition.

In a particular embodiment, the perfume composition comprising the pro-perfume compound is not dispersed in or absorbed within the carrier.

In a particular embodiment, the dry shampoo composition may further comprise a conditioning agent.

In a particular embodiment, the conditioning agent may be a quaternary alkyl ammonium compound. The quaternary alkyl ammonium compound preferably comprises one nitrogen atom attached to 4 substitutents independently selected from linear or branched alkyl groups, preferably linear or branched C1 to C25 alkyl groups, or linear or brached alkenyl groups, preferably linear or branched C1 to C25 alkenyl groups. The quaternary alkyl ammonium compound may have a standard anionic counterion known in the field, such as a chloride, methosulfate or the like. The conditioning agent may preferably be selected from the group consisting of cetrimonium chloride, distearyldimonium chloride, behentrimonium chloride, behentrimonium methosulfate and any combination thereof.

In a particular embodiment, the dry shampoo composition may comprise a conditioning agent in an amount of from 0.01 wt. % to 1.5 wt. %, preferably of from 0.1 wt. % to 1 wt. %, based on the total weight of the dry shampoo composition.

In a particular embodiment, the dry shampoo composition comprises an anticaking agent. An anticaking agent is understood as an additive placed in powdered or granulated materials to prevent the formation of lumps (caking) and for easing packaging, transport, flowability, and consumption

In a particular embodiment, the anticaking agent comprises tricalcium phosphate, powdered cellulose, magnesium stearate, sodium bicarbonate, sodium ferrocyanide, potassium ferrocyanide, calcium ferrocyanide, bone phosphate (i.e. Calcium phosphate), sodium silicate, silicon dioxide, calcium silicate, magnesium trisilicate, talcum powder, sodium aluminosilicate, potassium aluminium silicate, calcium aluminosilicate, bentonite, aluminium silicate, stearic acid, polydimethylsiloxane and silica, preferably magnesium stearate.

In a particular embodiment, the dry shampoo composition comprises the anticaking agent in an amount of from 0.1 wt. to 10 wt. %, preferably from 1 wt. % to 6 wt. %, based on the total weight of the dry shampoo composition.

In a particular embodiment, the dry shampoo composition may further comprise a moisturizing agent. A moisturizing agent is herein understood as a cosmetic ingredient for protecting, moisturizing, and lubricating the skin or hair.

In a particular embodiment, the moisturizing agent may comprise cetyl alcohol, CARBOPOL® 934, ethoxylated castor oil, paraffin oils, lanolin and its derivatives, silicone compounds such as silicone surfactants like DC® 3225C (Dow Corning) and/or silicone emollients, silicone oil (DC-200® ex. Dow Corning), propylene glycol, dipropylene glycol, glycerin, silicone, glycols, botanical extracts, and esters, preferably glycerin.

In a particular embodiment, the dry shampoo composition comprises the moisturizing agent in an amount of from 0.01 wt. to 10 wt. %, preferably from 0.1 wt. % to 6 wt. %, based on the total weight of the dry shampoo composition.

In a particular embodiment, the dry shampoo composition may further comprise a conditioning composition.

In a particular embodiment, the conditioning composition comprises a vegetable oil, preferably vegetable oil with conditioning properties, such as olive oil, soy bean oil, macadamia oil, argan oil, acovado oil and/or sweet almond oil, natural extracts, preferably natural extracts with conditioning properties, such as cocos nicifera fruit extract, glycin soja extract, cocoyl hydrolized keratine, paraffinum liquidum, castor oil or any combination thereof.

In a particular embodiment, the dry shampoo composition comprises the conditioning composition in an amount of from 0.01 wt. to 10 wt. %, preferably from 0.1 wt. % to 6 wt. %, based on the total weight of the dry shampoo composition.

In a particular embodiment, the dry shampoo composition may further comprise a benefit agent.

In a particular embodiment, the benefit agent may comprise retinoids such as retinol, Vitamin E, actives for stimulating hair growth known to a skilled person, actives for healing itchy scalp, actives for providing a fresh effect, such as caffeine, capsaicin, tea tree oil, menthol, methyl lactate.

In a particular embodiment, the dry shampoo composition comprises the benefit agent in an amount of from 0.001 wt. to 10 wt. %, preferably from 0.005 wt. % to 6 wt. %, preferably 0.01 wt. % to 1 wt. %, based on the total weight of the dry shampoo composition.

In a particular embodiment, the dry shampoo composition further comprises a cationic, anionic, amphoteric or non-ionic surfactant, preferably a cationic surfactant.

In a particular embodiment, the dry shampoo composition does not comprise a surfactant such as e.g. sodium dodecylbenzensulfonate. In view of the presence of a dry shampoo active material as defined above, surfactants are not necessarily needed in the dry shampoo composition.

In a particular embodiment, the dry shampoo composition does not comprise bleaching agents. Bleaching agents or optical brighteners are typically not desirable in dry shampoo compositions. Examples of bleaching agents are sodium carbonate peroxide and disodium anilinomorpholinotriazinylaminostilbenesulfonate.

In a particular embodiment, the dry shampoo composition may be in form of a powder or a suspension.

The present invention further relates to a dry shampoo article comprising
a dry shampoo composition as described hereinabove and
optionally, a propellant.

In a particular embodiment, the dry shampoo article may be in form of a powder, a liquid or an aerosol.

In a particular embodiment, the dry shampoo article is provided as an aerosol and may comprise at least one propellant. An aerosol dry shampoo article is herein understood as a composition comprising a dry shampoo composition and a propellant.

When a dry shampoo composition is provided as an aerosol the dry shampoo composition is applied in a diffusive manner, and may increase the transparency of the dry shampoo composition when compared to a powder application. After spraying the aerosol dry shampoo composition onto the hair, the propellant comprised in the dry shampoo article evaporates and a dry powder remains on the hair.

In a particular embodiment, the propellant may comprise butane, isobutane, propane, liquefied petroleum gas, dimethyl ether, trichlorofluoromethane, dichiorodiiluoroiriethane, dichlorotetrafluorothane, monochlorodifluoro methane, trichlorotrifluoroethane propane, carbon dioxide, nitrous oxide, or any combination combination thereof. In a particular embodiment, the propellant is a combination of butane, isobutane and propane.

In a particular embodiment, the dry shampoo article may comprise the propellant in an amount of from 75 wt. % to 93 wt. %, preferably from 80 wt. % to 90 wt. %, more preferably from 84 wt. % to 87 wt. %, based on the total weight of the dry shampoo article.

In a particular embodiment, the dry shampoo article may comprise the dry shampoo composition in an amount of from 7 wt. % to 25 wt. %, preferably from 10 wt. % to 20 wt. %, more preferably from 13 wt. % to 16 wt. %, based on the total weight of the dry shampoo article.

The present invention further relates to a use of a dry shampoo composition as described hereinabove or the dry shampoo article as described herein-above to confer, improve, enhance, modify or prolong the fragrance intensity of the perfume composition on hair or in the air surrounding the hair of a mammal over a prolonged period of time, preferably over a period of time of 18 hours, preferably 24 hours, more preferably 48 hours under ambient conditions.

The present invention further relates to a method for conferring, improving, enhancing, modifying or prolonging the fragrance intensity of the perfume composition on hair or in the air surrounding the hair of a mammal over a prolonged period of time, preferably over a period of time of 18 hours, preferably 24 hours, more preferably 48 hours, under ambient conditions, wherein the method comprises the step of applying the dry shampoo composition as described herein-above or the dry shampoo article as described herein-above to the hair of the mammal.

The dry shampoo composition may be applied by sprinkling into the hair or maybe sprinkled into a user's hand and then applied to the hair. The dry shampoo composition can be in the form of an aerosol which can be sprayed onto the hair of a user. The applied composition can be distributed through the hair by messaging into the hair, for example, in order to remove grease, oil and other undesirable elements from soiled hair.

The present invention further relates to a use of a pro-perfume compound as described herein-above to confer, improve, enhance, modify or prolong the fragrance intensity of dry shampoo composition or a dry shampoo article.

The present invention further relates to a method of conferring, improving, enhancing, modifying or prolonging the fragrance intensity of a dry shampoo composition or dry shampoo article, the method comprises the step of adding a pro-perfume as described herein-above to the dry shampoo composition or dry shampoo article.

### Examples

### Preparation and Evaluation of a Dry Shampoo Composition According to the Present Invention

a) Dry shampoo composition

| | Ingredients/INCI name | % |
|---|---|---|
| A | Alcohol Denat. ¹⁾ | 62.70 |
| B | Magnesium Stearate ²⁾ | 3.40 |
| | Cetrimonium Chloride ³⁾ | 0.5 |
| | Aluminum Starch | 33.40 |
| | Octenylsuccinate ⁴⁾ | |
| | TOTAL: | 100 |

¹⁾ ETHYL ALCOHOL FLAVOUR GRADE, origin: Firmenich SA
²⁾ MAGNESIUM STEARATE, origin: Firmenich SA
³⁾ GENAMIN CTAC, origin: Clariant
⁴⁾ AGENAFLO OS 9051, origin: Agrana

b) Perfume composition A

### Composition of Fragrance A

| Perfume ingredients | % |
|---|---|
| 3,7-dimethyl-3-octanol | 54.64% |
| 2-phenylethanol | 3.46% |
| 2-ethyl-3-hydroxy-4(4h)-pyranone | 5.80% |
| (−)-propyl (2S)-2-[(2-methyl-2- | 1.35% |
| butanyl)oxy]propanoate | |
| methyl 2-aminobenzoate | 1.26% |
| 3-ethoxy-4-hydroxybenzaldehyde | 14.25% |
| ethyl methylphenylglycidate | 0.23% |
| 2-(6,6-dimethylbicyclo[3.1.1]hept- | 2.45% |
| 2-en-2-yl)ethyl acetate | |
| 4-formyl-2-methoxyphenyl 2- | 1.83% |
| methylpropanoate | |
| 2-{1-[3,3- | 0.93% |
| dimethylcyclohexyl]ethoxy}-2- | |
| oxoethyl propionate | |
| 2-{1-[3,3- | 6.01% |
| dimethylcyclohexyl]ethoxy}-2- | |
| methylpropyl propionate | |
| 1-oxa-12-cyclohexadecen-2-one | 0.92% |
| and 1-oxa-13-cyclohexadecen-2-OnE | |
| 1,4-dioxacycloheptadecane-5,17-dione | 6.40% |
| 2/3/4-(5,5,6- | 0.46% |
| trimethylbicyclo[2.2.1]hept-2-yl)-1- | |
| cyclohexanol (a/b/c) + 2-(1,7,7- | |
| trimethylbicyclo[2.2.1]hept-2-yl)-1- | |
| cyclohexanol (d) | |
| sum | 100.00% |

c) Preparation

Phase A was weighted. Under gentle agitation, ingredients of phase B were added, one by one and by small portion. The mixture was stirred until homogenous. Then the mixture was put in the proper can and the propellant gas was added (Propane/Butane at 3.2 bar and a ratio 14% base to 86% propellant gas).
d) Dry Shampoo wash protocol

All the aerosol manipulation have to be done under the hood. Before the preparation of the hair swatches, the aerosol cans were checked by shaking and actioning the spray for 2 seconds. The aerosol can was weighted and tared. 1.0 g. of dry shampoo was sprayed homogeneously on the hair swatch at a close distance of ˜10 cm from the hair. The can was weighted again to ensure that the proper amount of product was applied on the hair. After having weighted the can, if less than 1.0 g were applied on the hair, the application step was repeated. If more than 1.0 g was applied, the application step was repeated with a new hair swatch. Before evaluation, the hair swatch was left for 5-10 minutes at room temperature to ensure it was completely dry.
e) Evaluation on Hair

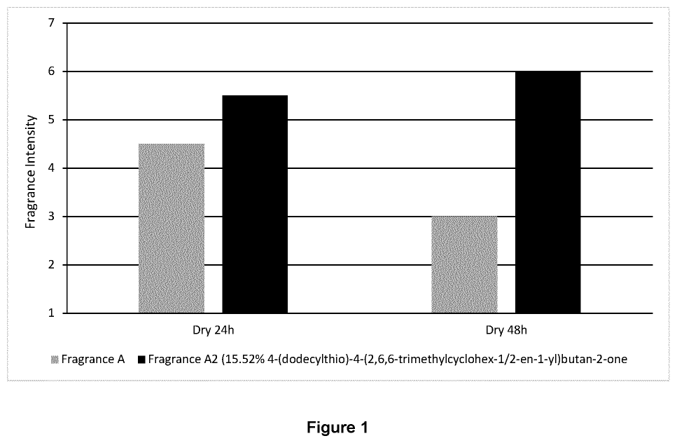

Fragrance intensity of the hair swatches was evaluated after 24 h and 48 h according to the following perfume intensity scale 1—Imperceptible, 2—Slightly perceptible, 3—Weak, 4—Medium, 5—Sustained, 6—Intense, 7—Very intense. Throughout the washing protocol, hands were protected by gloves. The results are shown in the Table below and in FIG. 1.

| | | | Intensity | |
|---|---|---|---|---|
| | | Dosage in | Dry | Dry |
| Product | Base | application | 24 h | 48 h |
| Fragrance A | Dry Shampoo | 0.14% | 4.50 | 3.00 |
| | composition | | | |
| Fragrance A2 (+15.52% 4- | Dry Shampoo | 0.14% | 5.50 | 6.00 |
| (dodecylthio)-4-(2,6,6- | composition | | | |
| trimethylcyclohex-2-en-1- | | | | |
| yl)butan-2-one) | | | | |

Fragrance A2 = Fragrance A + 15.52% 4-(dodecylthio)-4-(2,6,6-trimethylcyclohex-2-en-1-yl)butan-2-one

Intensity evaluated on a scale from 1-7 where:
1—Imperceptible
2—Slightly perceptible
3—Weak
4—Medium
5—Sustained
6—Intense
7—Very intense

1. A dry shampoo composition comprising
a perfume composition comprising a pro-perfume compound,
a dry shampoo active material and
optionally, a carrier material.

2. The dry shampoo composition according to claim 1, wherein the pro-perfume compound is an oxygen and/or water sensitive pro-perfume compound.

3. The dry shampoo composition according to claim 1, wherein the pro-perfume compound is a compound of formula 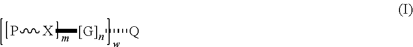
wherein:
a) w represents an integer from 1 to 10000;
b) n represents 1 or 0;
c) m represents an integer from 1 to 6;
d) P represents a hydrogen atom or a radical susceptible of generating an odoriferous α,β-unsaturated ketone, aldehyde or carboxylic ester and is represented by the formula 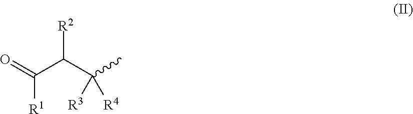
in which the wavy line indicates the location of the bond between said P and X;
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent, independently of each other, a hydrogen atom, an aromatic ring, or a C₁ to C₁₅ linear, cyclic or branched alkyl, alkenyl or alkadienyl radical, optionally substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being optionally substituted by C₁ to C₈ linear, branched or cyclic alkyl or alkenyl groups; and with the proviso that at least one of the P groups is of the formula (II) as defined hereinabove;
e) X represents, independently from each other, a functional group selected from the group consisting of the formulae i) to xiv): 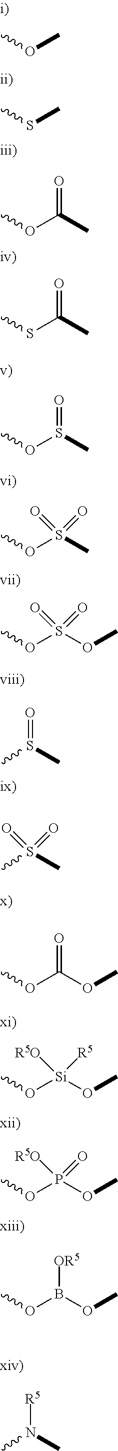
in which formulae the wavy lines are as defined previously and the bold lines indicate the location of the bond between said X and G, and R⁵ represents a hydrogen atom, a C₁ to C₂₂, saturated or unsaturated, alkyl group or an aryl group, optionally substituted by C₁ to C₆ alkyl or alkoxyl groups or halogen atoms; and with the proviso that X may not exist when P represents a hydrogen atom;
f) G represents a multivalent radical (with a m+1 valence) derived from cyclic, linear, alicyclic or branched alkyl, cyclic, linear or branched alkenyl, phenyl, alkylphenyl or alkenylphenyl hydrocarbon radical having from 1 to 22 carbon atoms, said hydrocarbon radical being optionally substituted and containing from 1 to 10 functional groups selected from the group consisting of halogens, alcohols, ethers, esters, ketones, aldehydes, carboxylic acids, thiols, thioethers, amines, quaternary amines and amides; and
g) Q represents a hydrogen atom (in which case w=1 and n=1), or represents a polymer or co-polymer selected from the group consisting of poly(alkylimine)s, polypeptides (e.g. polylysine) or polysaccharides selected from the group consisting of cellulose, cyclodextrins and starches, or cationic quaternised silicon polymers, or still a polymer or random co-polymer derived from monomeric units selected from the group consisting of formulae A) to C): 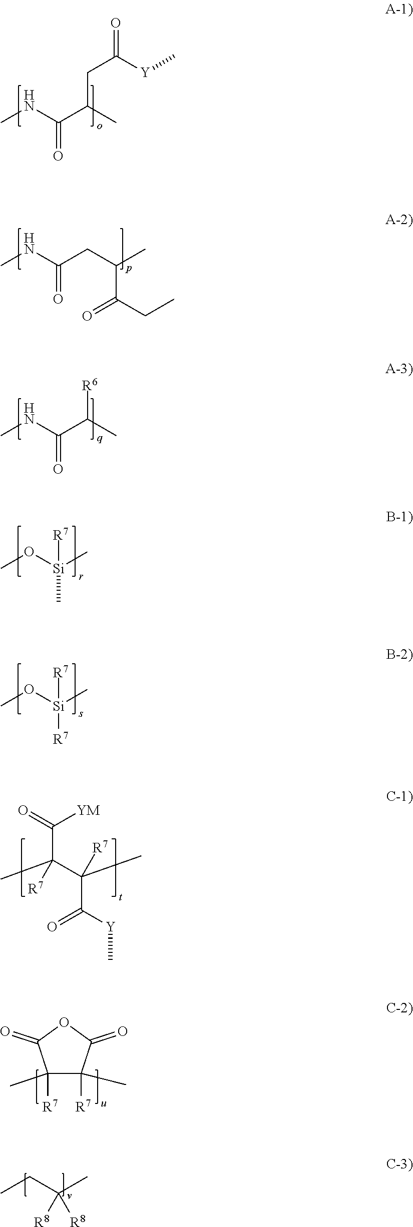
wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid;
R⁷ represents, simultaneously or independently, a hydrogen atom or a C₁-C₁₆ hydrocarbon group;
R⁸ represents, simultaneously or independently of each other
a hydrogen or halide atom;
a C₁-C₆ hydrocarbon group optionally comprising from 1 to 4 heteroatoms selected from the group consisting of oxygen and sulfur atoms;
a carboxylic group of formula COOR*, wherein R* represents a hydrogen atom, a C₁-C₆₀ alkyl or alkenyl group optionally comprising from 1 to 30 oxygen atoms;
a OR⁷ group or a COR⁷ group; or
a pyrrolidone unit, connected by the nitrogen atom; and
M represents a hydrogen atom, an alkali or earth alkali metal ion.

4. The dry shampoo composition according to claim 1, wherein the pro-perfume compound is a compound selected from the group consisting of formulae a) to d) 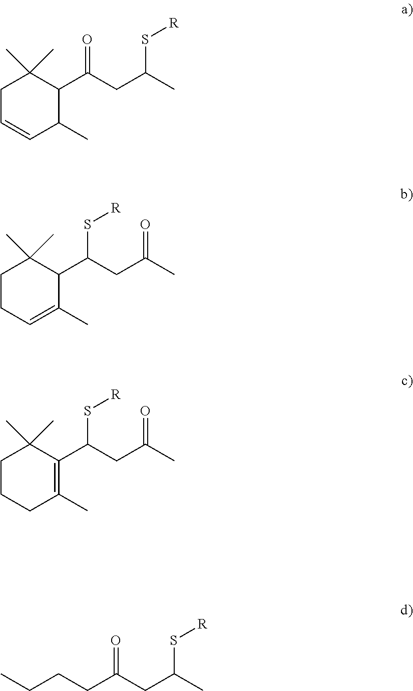
wherein R represents a C₁-C₂₀ alkyl or alkenyl group.

5. The dry shampoo composition according to claim 1, wherein the pro-perfume compound is a linear polysiloxane co-polymer comprising at least one repeating unit of formula 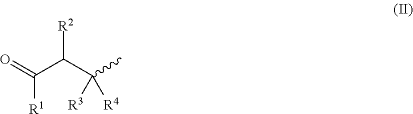
wherein the double hatched lines indicate the bonding to another repeating unit.

6. The dry shampoo composition according to claim 1, wherein the dry shampoo active material comprises a sebum or oil absorbent material.

7. The dry shampoo composition according to claim 1, wherein the dry shampoo active material comprises a starch material.

8. The dry shampoo composition according to claim 1, wherein the dry shampoo active material comprises a natural starch material selected from the group of cornstarch, potato starch, tapioca starch, rice starch, wheat starch, cassaya starch, a modified starch material selected from the group consisting of aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch and sodium starch glycolate, or any combination thereof.

9. The dry shampoo composition according to claim 1, wherein the carrier material is a solvent.

10. The dry shampoo composition according to claim 1, further comprising a cationic, anionic, amphoteric or non-ionic surfactant.

11. Dry shampoo article comprising
a dry shampoo composition according to claim 1 and
optionally, a propellant.

12. (canceled)

13. A method for conferring, improving, enhancing, modifying or prolonging the fragrance intensity of the perfume composition on hair or in the air surrounding the hair of a mammal over a prolonged period of time, under ambient conditions, wherein the method comprises the step of applying the dry shampoo composition according to claim 1 to the hair of the mammal.

14. (canceled)

15. A method for conferring, improving, enhancing, modifying or prolonging the fragrance intensity of a dry shampoo composition or dry shampoo article, the method comprising the step of adding a pro-perfume according to claim 2 to the dry shampoo composition or dry shampoo article.

16. The dry shampoo composition according to claim 7, wherein the starch material is selected from a natural starch material or a chemically or enzymatically modified starch material, or any combination thereof.

17. The dry shampoo composition according to claim 9, wherein the solvent is ethanol.

18. The dry shampoo composition according to claim 1, further comprising a cationic surfactant.

19. The method according to claim 13, wherein the prolonged period of time is a period of time of 18 hours.

20. The method according to claim 13, wherein the prolonged period of time is a period of time of 24 hours.

21. The method according to claim 13, wherein the prolonged period of time is a period of time of 48 hours.
